(19) United States
(12) Patent Application Publication
(10) Pub. No.: US 2010/0297692 A1
(43) Pub. Date: Nov. 25, 2010

(54) REACTION MEDIUM FOR DETECTING AND/OR IDENTTIFYING STAPHYLOCCOUS AUREUS

(75) Inventors: Diane Halimi, St. Maurice de Beynost (FR); Sylvain Orenga, Neuville Sur Ain (FR); John Perry, Newcastle-Upon-Tyne (GB)

Correspondence Address: OLIFF & BERRIDGE, PLC, P.O. BOX 320850, ALEXANDRIA, VA, 22320-4850, US

(73) Assignee: BIOMERIEUX, MARCY L'ETOILE (FR)

(21) Appl. No.: 12/734,454

(22) PCT Filed: Nov. 25, 2008
(86) PCT No.: PCT/FR2008/052119

§ 371 Date: May 3, 2010

(30) Foreign Application Priority Data
Nov. 26, 2007 (FR) 0759309

(51) Int. Cl. C12Q 1/14

(52) U.S. Cl. 435/36

(57) ABSTRACT

A reaction medium for characterizing Staphylococcus aureus, that includes a metabolic indicator which is a beta-ribosidase substrate in combination with at least one other metabolic indicator and/or at least one metabolic regulator. A method for detecting and/or identifying Staphylococcus aureus, including:
providing a reaction medium that comprises a metabolic indicator which is a beta-ribosidase substrate in combination with at least one other metabolic indicator and/or at least one metabolic regulator;
inoculating the medium with a biological sample to be tested;
allowing for incubation; and
characterizing the presence of Staphylococcus aureus.

The present invention relates to a reaction medium specific for Staphylococcus aureus. It also relates to a method for detecting and/or identifying Staphylococcus aureus which uses such a medium.

Most species of the Staphylococcus genus are opportunistic pathogens in humans exhibiting a high risk in the event of a skin injury due to a trauma or due to direct implantation of a medical product.

Among the staphylococci, Staphylococcus aureus is unquestionably the most virulent species, since it produces a large number of extracellular enzymes and toxins. It can be responsible for numerous and varied pathological conditions, ranging from simple whitlow, to the most severe infections such as septicemia, endocarditis, pneumopathies or osteoarticular infections, for which the prognosis can be not very optimistic. It is often found in patients who must receive hospital care involving equipment such as syringes or catheters. There is therefore great value in detecting the presence of this pathogenic bacterium, which is increasingly implicated in nosocomial diseases.

In bacteriology, it is conventional to contrast the Staphylococcus aureus species, characterized by the production of a coagulase, with the other species termed “coagulase-negative”. The conventional methods for differentiating between Staphylococcus aureus and Staphylococcus non-aureus are based on the search for free coagulase and DNase and on obtaining agglutination on latex aimed at demonstrating the presence of fibrinogen affinity factor, protein A and capsular antigens. However, other, potentially pathogenic, staphylococcus species are capable of expressing a coagulase.

There are also techniques for culturing on selective media so as to make it possible to evaluate the presence of Staphylococcus aureus, such as the high-salt Chapman medium (containing 7.5% of NaCl), which is generally selective for Staphylococcus aureus and staphylococci which hydrolyze mannitol, and Baird Parker medium (containing potassium tellurite and lithium chloride as selective agents), which is used to isolate and count coagulase-positive staphylococci in food products and makes it possible to demonstrate a lecithinase activity. However, these techniques lack sensitivity (ability to demonstrate the species being sought when the latter is present in small amounts in a biological sample to be tested) and especially specificity (ability to detect the species being sought in the biological sample to be tested, containing other species) and give only a presumptive diagnosis requiring other confirmation tests. However, the additional handling necessary for the identification of Staphylococcus aureus increases the time and the cost of the analyses. They require a multitude of reagents and the involvement of qualified staff.

It is also possible to use culture media comprising enzyme substrates, in particular phosphatase and/or beta-glucosidase or alpha-glucosidase substrates. Mention may, ,for example, be made of the chromogenic culture medium CHROMagar (trademark) Staph. aureus which makes it possible to isolate staphylococci and to identify Staphylococcus aureus by means of chromogenic substrates which give a mauve coloration of the latter species (Gaillot O. et al., 2000. J. Clin. Microbiol. 38, 4, 1587-1591). The other species of the same genus are then detected by blue or colorless coloration, in theory. The identification is essentially based on phosphatase, beta-glucosidase, beta-glucuronidase and beta-galactosidase activities, and also on the presence of an inhibitor, deferoxamine, which makes it possible to differentiate Staphylococcus aureus and Staphylococcus epidermidis. However, the differentiation of Staphylococcus aureus and of Staphylococcus epidermidis is imperfect since the two species produce colonies of the same color on CHROMagar (trademark) medium. This is due to the lack of specificity of the phosphatase substrates, which are positive for Staphylococcus aureus and Staphylococcus epidermidis, and to the fact that the inhibition of the latter by deferoxamine is only partial.

It is also possible to use alpha-glucosidase substrates, as described in patent application WO 02/079486. Thus, using only the detection of alpha-glucosidase activity, it is possible to separate Staphylococcus aureus from Staphylococcus epidermidis and Staphylococcus saprophyticus, which are three of the main staphylococcus species isolated clinically. However, while alpha-glucosidase substrates with a chromophore having an indoxyl nucleus are very suitable for use in a solid medium, their use can be problematic in a liquid medium, since the coloration does not diffuse in the liquid medium.

Surprisingly, the inventors have demonstrated that the use of one or more ribosidase enzyme substrate(s) enables rapid, easy identification of Staphylococcus aureus. In particular, the use of one or more ribosidase enzyme substrate(s) in combination with a metabolic regulator and/or another metabolic indicator makes it possible to distinguish S. aureus from the other Staphylococcus species.

Before presenting the invention, the following definitions are given so as to enable the invention to be understood more clearly. They are in no way limiting.

The term reaction medium is intended to mean a medium comprising all the elements necessary for the expression of a metabolism and/or for the growth of microorganisms. The reaction medium may be solid, semi-solid or liquid. The term “solid medium” is intended to mean, for example, a gelled medium. Agar is the conventional gelling agent in microbiology for culturing microorganisms, but it is possible to use gelatin or agarose. A certain number of preparations are commercially available, such as, for example, Columbia agar, Trypticase-soy agar, MacConkey agar, Sabouraud agar or, more generally, those described in the Handbook of Microbiological Media (CRC Press).

The reaction medium may comprise one or more elements in combination, such as amino acids, peptones, carbohydrates, nucleotides, minerals, vitamins, etc. The medium may also comprise a colorant. By way of indication, as a colorant, mention may be made of Evans blue, neutral red, sheep blood, horse blood, an opacifier such as titanium oxide, nitroaniline, malachite green, brilliant green, one or more metabolic indicators, one or more metabolic regulators, etc.

The reaction medium may be a revealing medium, or a culture and revealing medium. In the first case, the microorganisms are cultured before inoculation and, in the second case, the detection and/or identification medium also constitutes the culture medium.

Those skilled in the art may also use a biplate, making it possible to readily compare two media, comprising various substrates or various selective mixtures, onto which the same biological sample will have been deposited.

The term “metabolic regulator” is intended to mean any compound(s) which regulate(s) the growth of a microorganism. This metabolic regulator may in particular be an osidase regulator, a selective compound, such as in particular lithium chloride, potassium tellurite or sodium chloride, or a mixture of selective compounds, an antibiotic, a mixture of antibiotic(s) and/or antifungal agent(s) optionally comprising one or more beta-lactams, one or more aminoglycosides, one or more glycopeptides, one or more quinolones, one or more polypeptides, amphotericin, one or more azole compounds, which makes it possible to promote the detection of Staphylococcus aureus, inhibitors which promote the growth of Staphylococcus aureus bacteria, such as, in particular, lithium chloride (LiCl), sodium azide (NaN₃), colistin, amphotericin, aztreonam; colimycin, sodium chloride (NaCl), deferoxamine, and vibriostatic compound O/129.

Preferably, the medium according to the invention comprises a selective mixture for promoting the growth of Staphylococcus aureus and/or inhibiting the growth of the other species of microorganisms. This mixture preferably comprises lithium chloride, a vibriostatic compound O/129, aztreonam and amphotericin.

The term “metabolic osidase regulator” is intended to mean in particular compounds which can induce or repress the expression of one or more osidase activities in the reaction medium.

Preferably, the concentration of metabolic regulator is between 0.5 mg/l and 75 g/l.

The term “metabolic indicator” is intended to mean any compound(s) which make(s) it possible to demonstrate the presence of a microorganism. This metabolic indicator may in particular be a substrate, such as a ribosidase substrate, or a substrate other than a beta-ribosidase substrate, such as, in particular, an osidase substrate, an esterase substrate or a peptidase substrate, and more particularly a beta-glucosidase and/or beta-galactosidase and/or beta-glucuronidase and/or phosphatase substrate, thereby promoting the detection of S. aureus. The medium may also comprise one or more metabolic indicators different than a beta-ribosidase substrate, such as a combination of substrates. Those skilled in the art will adjust the concentration of substrate(s) according to the microorganism that it is desired to identify. Preferably, the concentration of substrate is between 5 and 1000 mg/l, preferably between 50 and 400 mg/l.

The term substrate is intended to mean a molecule that can be hydrolyzed by an enzyme, such as beta-ribosidase, so as to give a product enabling the direct or indirect detection of a microorganism. This substrate comprises in particular a first part specific for the enzyme activity to be revealed and a second part which acts as a label, hereinafter referred to as label part. This label part may be chromogenic, fluorogenic, luminescent, etc.

The term “beta-ribosidase substrate” is intended to mean, in particular, 2-hydroxyphenyl-β-D-riboside (catechol-β-D-riboside); magenta-β-D-riboside (5-bromo-6-chloro-3-indoxyl-β-D-riboside); dihydroxyflavone-β-D-riboside; X-β-D-riboside (5-bromo-4-chloro-3-indoxyl-β-D-riboside); pink-β-D-riboside (6-chloro-3-indoxyl-β-D-riboside); 6-bromo-3-indoxyl-β-D-riboside; 5-bromo-3-indoxyl-β-D-riboside; 6-fluoro-3-indoxyl-β-D-riboside; alizarin-β-D-riboside; (P)-nitrophenyl-β-D-riboside; 4-methylumbelliferyl-β-D-riboside; naphthol-β-D-riboside; dichloroaminophenyl-β-D-riboside.

Preferably, the concentration of beta-ribosidase substrate in the medium according to the invention is between 25 and 1000 mg/l and more preferably between 50 and 400 mg/l.

The term “beta-glucosidase substrate” is intended to mean, in particular, 2-hydroxyphenyl-β-D-glucoside (catechol-β-D-glucoside); magenta-β-D-glucoside (5-bromo-6-chloro-3-indoxyl-β-D-glucoside); dihydroxy-flavone-β-D-glucoside; X-β-D-glucoside (5-bromo-4-chloro-3-indoxyl-β-D-glucoside); pink-β-D-glucoside (6-chloro-3-indoxyl-β-D-glucoside); 6-bromo-3-indoxyl-β-D-glucoside; 5-bromo-3-indoxyl-β-D-glucoside; 6-fluoro-3-indoxyl-β-D-glucoside; alizarin-β-D-glucoside; (P)-nitrophenyl-β-D-glucoside; 4-methylumbelliferyl-β-D-glucoside; naphtholbenzein-β-D-glucoside; indoxyl-N-methyl-β-D-glucoside; 5-bromo-4-chloro-3-indoxyl-N-methyl-β-D-glucoside; 8-hydroxyquinoline-β-D-glucoside; naphthol-β-D-glucoside.

Preferably, the concentration of beta-glucosidase substrate in the medium according to the invention is between 25 and 1000 mg/l and more preferably between 50 and 400 mg/l.

The term “beta-galactosidase substrate” is intended to mean, in particular, 2-hydroxyphenyl-β-D-galactoside (catechol-β-D-galactoside); magenta-β-D-galactoside (5-bromo-6-chloro-3-indoxyl-β-D-galactoside); dihydroxyflavone-β-D-galactoside; X-β-D-galactoside (5-bromo-4-chloro-3-indoxyl-β-D-galactoside); pink-β-D-galactoside (6-chloro-3-indoxyl-β-D-galactoside); 6-bromo-3-indoxyl-β-D-galactoside; 5-bromo-3-indoxyl-β-D-galactoside; 6-fluoro-3-indoxyl-β-D-galactoside; alizarin-β-D-galactoside; (P)-nitrophenyl-β-D-galactoside; 4-methylumbelliferyl-β-D-galactoside; naphtholbenzein-β-D-galactoside; indoxyl-N-methyl-β-D-galactoside; 5-bromo-4-chloro-3-indoxyl-N-methyl-β-D-galactoside; 8-hydroxyquinoline-β-D-galactoside; naphthol-β-D-galactoside.

Preferably, the concentration of beta-galactosidase substrate in the medium according to the invention is between 25 and 1000 mg/l and more preferably between 50 and 400 mg/l.

The term Staphylococcus aureus is intended to mean any strain of Staphylococcus aureus, including the resistant strains such as MRSAs (methicillin-resistant Staphylococcus aureus), MSSAs, GISAs, VISAs or VRSAs (glycopeptide-intermediate, vancomycin-intermediate or vancomycin-resistant Staphylococcus aureus, respectively).

The term biological sample is intended to mean a clinical sample, derived from a specimen of biological fluid, or a food sample, derived from any type of food. This sample may thus be liquid or solid and mention may be made, in a nonlimiting manner, of a clinical blood, plasma, urine or feces sample, nose, throat, skin, wound or cerebrospinal fluid specimens, a food sample from water, from drinks such as milk or a fruit juice, from yogurt, from meat, from eggs, from vegetables, from mayonnaise, from cheese; from fish, etc., a food sample derived from an animal feed, such as, in particular, a sample derived from animal meals.

In this respect, the invention relates to a reaction medium for characterizing Staphylococcus aureus, that comprises a metabolic indicator which is a beta-ribosidase substrate in combination with at least one other metabolic indicator and/or at least one metabolic regulator.

The invention also relates to the use of a metabolic indicator which is a beta-ribosidase substrate in combination with another metabolic indicator and/or a metabolic regulator, for characterizing Staphylococcus aureus.

According to one preferred embodiment of the invention, said metabolic regulator is chosen from a regulator of osidase activity, a selective compound, such as, in particular, lithium chloride, potassium tellurite or sodium chloride, or a mixture of selective compounds, an antibiotic or a mixture of antibiotic(s) and/or antifungal agent(s), such as, in particular, one or more beta-lactams, one or more aminoglycosides, one or more glycopeptides, one or more quinolones, one or more polypeptides, amphotericin and azole compounds, alone or in combination with one or more selective compounds.

According to one preferred embodiment of the invention, said other metabolic indicator is an enzyme substrate, preferably an osidase, esterase, peptidase, beta-glucosidase and/or beta-galactosidase substrate. According to an even more preferred embodiment, said other metabolic indicator is chosen from a beta-glucosidase and/or beta-galactosidase substrate.

According to one preferred embodiment of the invention, said beta-ribosidase substrate is chosen from 2-hydroxyphenyl-β-D-riboside (catechol-β-D-riboside); magenta-β-D-riboside (5-bromo-6-chloro-3-indoxyl-β-D-riboside); dihydroxyflavone-β-D-riboside; X-β-D-riboside (5-bromo-4-chloro-3-indoxyl-β-D-riboside); pink-β-D-riboside (6-chloro-3-indoxyl-β-D-riboside); 6-bromo-3-indoxyl-β-D-riboside; 5-bromo-3-indoxyl-β-D-riboside; 6-fluoro-3-indoxyl-β-D-riboside; alizarin-β-D-riboside; (P)-nitrophenyl-β-D-riboside; 4-methyl-umbelliferyl-β-D-riboside.

According to one preferred embodiment of the invention, said beta-ribosidase substrate is catechol-β-D-riboside.

According to another preferred embodiment of the invention, said beta-ribosidase substrate is X-β-D-riboside (5-bromo-4-chloro-3-indoxyl-β-D-riboside).

According to one preferred embodiment of the invention, said reaction medium is a solid medium.

According to another preferred embodiment of the invention, said reaction medium is a liquid medium. In this case, said beta-ribosidase substrate is preferably catechol-(3-D-riboside since it allows the detection/identification of S. aureus in approximately 18h.

The invention also relates to a method for detecting and/or identifying Staphylococcus aureus, characterized in that it comprises or consists of the following steps:
a) providing a reaction medium as defined above,
b) inoculating the medium with a biological sample to be tested,
c) allowing for incubation, and
d) characterizing the presence of Staphylococcus aureus.

This method may also comprise a confirmation step. This confirmation step may in particular be a biochemical, immunological or molecular identification.

The inoculation of the microorganisms can be carried out by any of the inoculation techniques known to those skilled in the art. An incubation step can be carried out at a temperature for which the expression of the enzyme activity that it is desired to detect is optimal, which those skilled in the art can easily choose according to the enzyme activity to be detected.

According to one preferred embodiment of the invention, in step c), the period allowing for incubation is between 16 and 48 h, preferably between 18 and 24 h.

Step d) can be carried out by means of a visual examination, by colorimetry or by fluorimetry.

The S. aureus strains with acquired resistance to one or more antibiotics, and in particular MRSAs (methicillin-resistant S. aureus strains), GISAs (glycopeptide-intermediate S. aureus strains), VISAs (vancomycin-intermediate S. aureus strains), VRSAs (vancomycin-resistant S. aureus strains) and other resistant phenotypes can be detected by adding the appropriate antibiotic, chosen in particular from β-lactams, cephalosporins, glycopeptides, amino-glycosides, quinolones, polypeptides, etc.

The examples below are given by way of explanation and are in no way limiting in nature. They will make it possible to understand the invention more clearly.

### EXPERIMENT 1

### Evaluation of β-D-Ribosidase Substrates for Characterizing S. aureus:

The media hereinafter were prepared in 200 ml of a Columbia agar base (Oxoid), in combination with the chromogenic β-D-ribosidase substrates. The various substrates and also the concentrations and the additives used in the media are described in Table 1 below:

TABLE 1
Abbreviations and concentrations of the chromogenic β-D-ribosidase substrates used

| | | Concentration | Additives and |
|---|---|---|---|
| Chromogenic substrates | Abbreviation used | in mg/l | concentration |
| 2-Hydroxyphenyl-beta-D-riboside | Catechol-β-D-riboside | 400 | Ammoniacal iron |
| | | | citrate 100 mg/l |
| 5-Bromo-6-chloro-3-indoxyl-beta-D- | Magenta-β-D-riboside | 100 | — |
| riboside | | | |
| Dihydroxyflavone-beta-D-riboside | DHF-β-D-riboside | 400 | Ammoniacal iron |
| | | | citrate 100 mg/l |
| 5-Bromo-4-chloro-3-indoxyl-beta-D- | X-β-D-riboside | 80 and 200 | — |
| riboside | | | |
| 6-Chloro-3-indoxyl-beta-D-riboside | Pink-β-D-riboside | 100 and 200 | — |
| 4-Methylumbelliferyl-beta-D-riboside | 4-MU-β-D-riboside | 50 | — |

The β-D-ribosidase substrates are solubilized in DMSO and added to the media so as to obtain the concentrations indicated in Table 1.

191 bacterial strains were inoculated onto each of the media using a suspension at 0.5 MacFarland diluted to 1/1000. The dishes were incubated at 37° C. and then read.

The media were distributed into Petri dishes, then inoculated with approximately 100 000 CFU, then incubated at 37° C. for 48 h. The colonies formed were examined visually after incubation times of 18, 24 and 48 hours.

The stock collection of 191 strains comprised: 79 MRSAs, 48 Staphylococcus spp, 61 Enterococcus spp and 3 alpha-hemolytic Streptococcus.

The results obtained are indicated in the table below:

TABLE 2
Evaluation of the various β-D-ribosidase substrates

| | | | | | | | | 4-MU-β- | | | Magenta-β- | | |
|---|---|---|---|---|---|---|---|---|---|---|---|---|---|
| | | X-β-riboside | | | | | | riboside | | | riboside | | |
| | | 80 mg/L | | | 200 mg/L | | | 50 mg/L | | | 100 mg/L | | |
| | | % of strains producing colored or fluorescent colonies | | | | | | | | | | | |
| | Total | 18 h | 24 h | 48 h | 18 h | 24 h | 48 h | 18 h | 24 h | 48 h | 18 h | 24 h | 48 h |
| S. aureus | 79 | 84 | 92 | 97 | 82 | 87 | 95 | 97 | 97 | 97 | 71 | 85 | 95 |
| (MRSA) | | | | | | | | | | | | | |
| S. aureus | 14 | 64 | 100 | 100 | 64 | 64 | 100 | 100 | 100 | 100 | 64 | 64 | 100 |
| (MSSA) | | | | | | | | | | | | | |
| Coagulase- | 34 | 3 | 6 | 6 | 3 | 3 | 3 | 82 | 82 | 82 | 0 | 3 | 6 |
| negative | | | | | | | | | | | | | |
| Staphylococci | | | | | | | | | | | | | |
| Micrococcus | 2 | 0 | 0 | 0 | 0 | 0 | 0 | 0 | 0 | 0 | 0 | 0 | 0 |
| luteus | | | | | | | | | | | | | |
| Enterococci | 61 | 2 | 2 | 2 | 2 | 2 | 2 | 57 | 57 | 49 | 0 | 2 | 2 |
| Alpha- | 3 | 0 | 0 | 0 | 0 | 0 | 0 | 0 | 0 | 0 | 0 | 0 | 0 |
| hemolytic | | | | | | | | | | | | | |
| streptococci | | | | | | | | | | | | | |

| | | | | | | | Catechol-β- | | | DHF-β- | | |
|---|---|---|---|---|---|---|---|---|---|---|---|---|
| | Pink-β-riboside | | | | | | riboside | | | riboside | | |
| | 100 mg/L | | | 200 mg/L | | | 400 mg/L | | | 400 mg/L | | |
| | % of strains producing colored or fluorescent colonies | | | | | | | | | | | |
| | 18 h | 24 h | 48 h | 18 h | 24 h | 48 h | 18 h | 24 h | 48 h | 18 h | 24 h | 48 h |
| S. aureus | 91 | 96 | 100 | 84 | 94 | 100 | 100 | 100 | 100 | 100 | 100 | 100 |
| (MRSA) | | | | | | | | | | | | |
| S. aureus | 64 | 64 | 100 | 57 | 64 | 100 | 100 | 100 | 100 | 100 | 100 | 100 |
| (MSSA) | | | | | | | | | | | | |
| Coagulase- | 3 | 3 | 3 | 3 | 3 | 6 | 3 | 3 | 3 | 68 | 68 | 91 |
| negative | | | | | | | | | | | | |
| Staphylococci | | | | | | | | | | | | |
| Micrococcus | 0 | 0 | 0 | 0 | 0 | 0 | 0 | 0 | 0 | 0 | 0 | 0 |
| luteus | | | | | | | | | | | | |
| Enterococci | 0 | 0 | 2 | 0 | 0 | 0 | 2 | 2 | 2 | 30 | 30 | 48 |
| Alpha- | 0 | 0 | 0 | 0 | 0 | 0 | 0 | 0 | 0 | 0 | 0 | 0 |
| hemolytic | | | | | | | | | | | | |
| streptococci | | | | | | | | | | | | |

All the media clearly enabled the detection of Staphylococcus aureus.

The reaction media comprising the substrates catechol-β-D-riboside or X-β-riboside are most specific and most sensitive since they allowed good detection of Staphylococcus aureus (MRSA and MSSA), after only 18 h of incubation for catechol-β-D-riboside and 24 h of incubation for X-β-D-riboside, with only 3% of false positives among the coagulase-negative staphylococci.

The medium containing DHF-β-D-riboside was also very sensitive, but less specific, since the percentage of coagulase-negative staphylococci that were β-ribosidase-positive was 68% at 24 hours and 91% at 48 hours.

### EXPERIMENT 2

### Evaluation of Catechol-β-D-Riboside in a Liquid Medium

The medium hereinafter had the same composition as a chromID S. aureus base (bioMérieux), with no selective agents nor agar, so as to obtain a liquid medium. The substrates and inducers were replaced, before autoclaving, with 300 ml/g of catechol-β-D-riboside, solubilized in DMSO, and with ammoniacal iron citrate at 500 mg/l. The medium was then distributed into tubes in a proportion of 1.5 ml/tube.

For this medium, the inoculation was carried out using precultures prepared for 24 h at 37° C. A suspension of physiological saline at 0.5 McF was prepared and then each tube was inoculated with 10 μl of bacterial suspension.

The readings were carried out after 24 and 48 hours of incubation at 37° C.

In total, 11 staphylococcus strains were tested; 5 S. aureus and 6 of other species of the Staphylococcus genus (coagulase-negative). The results obtained have been recorded in Table 3 below:

TABLE 3
Evaluation of catechol-β-D-riboside in a
liquid medium

| | | Catechol-β-D-riboside | | |
|---|---|---|---|---|
| | | Number of strains | | |
| | | producing a coloration | | |
| | Total | Positive | Positive | Positive |
| Strains | number | at 18 h | at 24 h | at 48 h |
| S. aureus (MRSA and | 5 | 5/5 | 5/5 | 5/5 |
| MSSA) | | | | |
| Coagulase-negative | 6 | 0/6 | 0/6 | 1/6 |

The results obtained in a solid medium and showing the high specificity of catechol-β-D-riboside for S. aureus were confirmed in a liquid medium: 100% coloration for S. aureus was observed from 18 hours onward, whereas none of the other species of the Staphylococcus genus were detected.

### EXPERIMENT 3

### Confirmation of the Results Obtained with the Substrates Catechol-β-D-Riboside and X-β-D-Riboside on a Broad Panel of Staphylococcus spp

In order to confirm the relevance of the reaction media comprising the substrate catechol-β-D-riboside or the substrate X-β-D-riboside, the following experiments were also carried out:

Catechol-β-D-riboside: A GTS medium (bioMérieux) supplemented with 300 mg/l of catechol-β-D-riboside, then autoclaved, and with 500 mg/l of ammoniacal iron citrate was used and distributed into Petri dishes 55 mm in diameter.

X-β-D-riboside: A modified Chapman medium, without NaCl, autoclaved and supplemented with 60 mg/l of X-β-D-riboside, was used and distributed into Petri dishes 55 mm in diameter.

For the two media, the inoculation was carried out using precultures prepared for 24 h at 37° C. A suspension in physiological saline at 0.5 McF was prepared and then each strain was inoculated onto a dish using a 10 μl calibrated loop.

The readings were carried out after 24 and 48 hours of incubation at 37° C.

In total, 90 Staphylococci were tested: 34 S. aureus, including 25 MRSAs, and 46 other species of Staphylococcus (including S. schleiferi; S. capitis; S. hyicus; S. intermedius; S. haemolyticus; S. auricularis; S. hominis; S. warneri; S. chromogenes; S. cohnii; S. epidermidis; S. sciuri; S. xylosus; S. saprophyticus).

The results obtained are recorded in Table 4.

TABLE 4
Evaluation of the substrates catechol-β-D-
riboside and X-β-D-riboside on a broad panel of
Staphylococcus spp. strains

| | Catechol-beta-D-riboside | | | | X-beta-D-riboside | | | |
|---|---|---|---|---|---|---|---|---|
| | Number of strains producing a coloration/total number of strains | | | | | | | |
| | Colony | | | | Colony | | | |
| Strains | color | positive at 18 h | positive at 24 h | positive at 48 h | color | positive at 18 h | positive at 24 h | positive at 48 h |
| S aureus | Grey | 19/34 | 31/34 | 34/34 | Green | 17/34 | 29/34 | 33/34 |
| (MRSA and | to | | | | to | | | |
| MSSA) | black | | | | turquoise | | | |
| Coag neg | | 10/46 | 14/46 | 14/46 | | 7/46 | 12/46 | 14/46 |

The results of this test show good specificity of these substrates for S. aureus, from 18 h onward. Specifically, of the 13 other species of the Staphylococcus genus, only 4 very minor species gave a coloration. These were the species Staphylococcus saprophyticus, Staphylococcus xylosus, Staphylococcus conhii and Staphylococcus sciuri, which, all 4 together, represent only 4.4% of the Staphylococcus strains found in clinical specimens, against 23.3% for Staphylococcus aureus (Kawamura et al., 1998, Distribution of Staphylococcus species among human clinical specimens and emended description of Staphylococcus caprae. J. Clin Microbial. 36: 2038-2042).

### EXPERIMENT 4

### Differentiation of Staphylococcus aureus with Respect to the Other Species of the Same Genus by Direct Detection of β-D-Ribosidase Activity and of Another Osidase Activity in a Gelled Medium

The media hereinafter were prepared in an autoclaved, modified Chapman medium, without NaCl, and combine a beta-ribosidase chromogenic substrate with a combination of one or two other metabolic indicator(s), i.e. one or two chromogenic substrate(s) other than that for detecting a β-ribosidase activity. These substrate combinations are given in the following table:

TABLE 5
Abbreviation of various combinations of
β-ribosidase chromogenic substrates and of chromogenic
substrates specific for other enzyme activities

| | β-D-ribosidase substrate | Substrate(s) combined |
|---|---|---|
| Abbreviation | [concentration in mg/l] | [concentration in mg/l] |
| A | X-β-D riboside | Pink-β-glucoside |
| | [60 mg/L] | [300 mg/L] |
| B | X-β-D riboside | Blue-β-galactoside |
| | [60 mg/L] | [600 mg/L] |
| C | X-β-D riboside | Pink-β-glucoside |
| | [100 mg/L] | [150 mg/L] |
| | | Blue-β-galactoside |
| | | [300 mg/L] |

The various substrates are solubilized in DMSO and added to the media.

For the media containing the substrate Blue-(3-galactoside, IPTG solubilized in osmosed water is added so as to obtain a concentration of 1 mg/l.

The media were distributed into Petri dishes 55 mm in diameter and the inoculation was carried out using precultures prepared for 24 h at 37° C. A suspension in physiological saline at 0.5 McF was prepared and then each strain was inoculated onto a dish using a 10 μl calibrated loop.

The readings were carried out after 18, 24 and 48 hours of incubation at 37° C.

In total, 28 Staphylococci strains were tested: 5 MRSAs and 3 S. aureus, 4 S. saprophyticus, 4 S. cohnii, 4 S. xylosus, 4 S. sciuri, 2 S. epidermidis and 2 S. haemolyticus.

In the table below, C represents the coloration of the colonies after incubation, I represents the strength of this coloration and IT defines the incubation time. The coloration strength is an arbitrary scale and can be defined in the following way:
The symbol “-” corresponds to an absence of activity
0.1 corresponds to the presence of a trace of coloration
0.5 corresponds to the presence of a very pale coloration
1 corresponds to the presence of a clear coloration of weak strength
1.5 corresponds to the presence of a coloration that is intermediate between colorations 1 and 2
2 corresponds to the presence of a clear coloration of medium strength
2.5 corresponds to the presence of a coloration that is intermediate between colorations 2 and 3
3 corresponds to the presence of a strong coloration
3.5 corresponds to the presence of a coloration that is intermediate between colorations 3 and 4
4 corresponds to the presence of a very strong coloration.

The results of the tests are given hereinafter:

TABLE 6
Evaluation of various combinations of substrates

| | | | A | | B | | C | |
|---|---|---|---|---|---|---|---|---|
| | Strains | IT | I | C | I | C | I | C |
| S. aureus | MRSA | 18 h | 2 | Turquoise | 3 | Turquoise | 2 | Turquoise |
| | 040 | 24 h | 2 | | 3 | | 3 | |
| | | 48 h | 3 | | 3 | | 3.5 | |
| | MRSA | 18 h | 1 | Turquoise | 2 | Turquoise | 1.5 | Turquoise |
| | 007 | 24 h | 2 | | 3 | | 2 | |
| | | 48 h | 3 | | 4 | | 3.5 | |
| | MRSA | 18 h | 2 | Turquoise | 1.5 | Turquoise | 0.5 | Turquoise |
| | 120 | 24 h | 2 | | 3 | | 1.5 | |
| | | 48 h | 3 | | 3 | | 3 | |
| | MRSA | 18 h | 2.5 | Turquoise | 2 | Turquoise | 0.1 | Turquoise |
| | 006 | 24 h | 2.5 | | 2 | | 2 | |
| | | 48 h | 3 | | 3 | | 3 | |
| | MRSA | 18 h | 2 | Turquoise | 3 | Turquoise | 2 | Turquoise |
| | 060 | 24 h | 2 | | 3 | | 2 | |
| | | 48 h | 3 | | 3 | | 3 | |
| | MSSA | 18 h | 3 | Turquoise | 3 | Turquoise | 2 | Turquoise |
| | 105 | 24 h | 3 | | 3 | | 2 | |
| | | 48 h | 3 | | 4 | | 3 | |
| | MSSA | 18 h | 0.1 | Turquoise | 3 | Turquoise | 0.5 | Turquoise |
| | 140 | 24 h | 1.5 | | 3 | | 1.5 | |
| | | 48 h | 3 | | 3 | | 3 | |
| | MSSA | 18 h | 0.1 | Turquoise | 0.1 | Turquoise | 0.5 | Turquoise |
| | 061 | 24 h | 1 | | 2 | | 2 | |
| | | 48 h | 3 | | 3 | | 3 | |
| Coag neg | S. sciuri | 18 h | 0.1 | Dark blue | — | — | — | — |
| | 051 | 24 h | 0.5 | violet | — | | — | |
| | | 48 h | 4 | | 3 | Turquoise | 2 | Dark blue |
| | S. sciuri | 18 h | 3 | violet | 2.5 | Turquoise | 2.5 | Blue/Violet |
| | 016 | 24 h | 4 | | 3 | | 2.5 | |
| | | 48 h | 4 | | 4 | | 2.5 | Dark blue |
| | S. sciuri | 18 h | 3 | violet | 3 | Turquoise | 3 | Dark blue |
| | 018 | 24 h | 4 | | 4 | | 3 | |
| | | 48 h | 4 | | 4 | | 3 | |
| | S. sciuri | 18 h | 1.5 | violet | 0.1 | Turquoise | 0.1 | Pink/Violet |
| | 141 | 24 h | 2 | | 0.5 | | 0.5 | |
| | | 48 h | 2 | | 3 | | 1.5 | |
| | S. saprophyticus | 18 h | — | — | 3 | Dark blue | 2 | Dark blue |
| | 153 | 24 h | — | | 3 | | 2.5 | |
| | | 48 h | 2 | Turquoise | 4 | | 3 | |
| | S. saprophyticus | 18 h | — | — | 3 | Dark blue | 2 | Dark blue |
| | 074 | 24 h | — | | 3 | | 2 | |
| | | 48 h | 1.5 | Turquoise | 4 | | 3 | |
| | S. saprophyticus | 18 h | — | — | 3 | Dark blue | 2 | Dark blue |
| | 152 | 24 h | 0.5 | Pink | 3 | | 2 | |
| | | 48 h | 3 | | 4 | | 3 | |
| | S. saprophyticus | 18 h | 0.1 | Turquoise | — | — | — | — |
| | 002 | 24 h | 0.1 | | — | | 0.1 | Turquoise |
| | | 48 h | 1.5 | | 2 | Turquoise | 3 | |
| | S. cohnii | 18 h | — | — | 1 | Dark blue | 0.1 | Dark blue |
| | 007 | 24 h | — | | 2 | | 1 | |
| | | 48 h | 0.5 | Turquoise | 4 | | 4 | |
| | S. cohnii | 18 h | — | — | — | — | — | Gray |
| | 039 | 24 h | — | | — | | — | |
| | | 48 h | 2 | Turquoise | 2 | Green | 0.1 | |
| | S. cohnii | 18 h | — | — | — | — | 0.5 | Pink |
| | 026 | 24 h | — | | — | | 1.5 | |
| | | 48 h | — | | — | | 3 | |
| | S. cohnii | 18 h | — | — | 2 | Dark blue | — | Pink |
| | 005 | 24 h | — | | 3 | | — | |
| | | 48 h | 2 | Turquoise | 4 | | 0.1 | |
| | S. xylosus | 18 h | 1.5 | Violet | 2 | Dark blue | 0.5 | Dark blue |
| | 038 | 24 h | 2 | | 3 | | 0.75 | |
| | | 48 h | 4 | | 4 | | 4 | |
| | S. xylosus | 18 h | 0.5 | Violet | 3 | Dark blue | 2 | Dark blue |
| | 067 | 24 h | 1.5 | | 4 | | 3 | |
| | | 48 h | 4 | | 4 | | 4 | |
| | S. xylosus | 18 h | 3 | Violet | 1 | Dark blue | 2 | Pink/Violet |
| | 008 | 24 h | 3 | | 1.5 | | 2 | |
| | | 48 h | 3 | | 4 | | 3 | |
| | S. xylosus | 18 h | 2 | Pink | — | — | — | Pink/Violet |
| | 009 | 24 h | 3 | | — | | 0.5 | |
| | | 48 h | 4 | Violet | 4 | Dark blue | 3 | |
| | S. epidermidis | 18 h | — | — | — | — | — | — |
| | 009 | 24 h | — | | — | | — | |
| | | 48 h | — | | 0.1 | Dark blue | 1 | Dark blue |
| | S. epidermidis | 18 h | — | — | — | — | — | — |
| | 025 | 24 h | — | | — | | — | |
| | | 48 h | — | | 0.1 | Dark blue | — | |
| | S. haemolyticus | 18 h | — | — | 0.5 | Dark blue | — | — |
| | 025 | 24 h | — | | 1.5 | | 1 | Dar blue |
| | | 48 h | — | | 4 | | 2 | |
| | S. haemolyticus | 18 h | 1.5 | Pink | — | — | — | — |
| | 028 | 24 h | 2 | | — | | 1.5 | Pink |
| | | 48 h | 4 | | — | | 2 | |

The results above show that it is possible to distinguish Staphylococcus aureus from the other species of staphylococci expressing a β-D-ribosidase from 18 hours of incubation onward with a combination of substrates.

Only one strain of Staphylococcus saprophyticus posed a problem at 48 hours, but there was no ambiguity at 24 hours of incubation, since the Staphylococcus aureus already had a good coloration strength.

### EXPERIMENT 5

### Detection of MRSAs by Means of their β-Ribosidase Activity in a Selective Medium

A modified Chapman medium, without NaCl, that had been autoclaved and supplemented with 100 mg/l of x-β-D-riboside and was selective, i.e. comprised metabolic inhibiting regulators (LiCl, vibriostatic compound O/129, aztreonam and amphotericin) for inhibiting MSSAs and most of the other Staphylococcus species, was used and distributed into Petri dishes 55 mm in diameter.

The substrate was solubilized in DMSO before being added to the medium.

The inoculation was carried out using precultures prepared for 24 h at 37° C. A suspension in physiological silane at 0.5 McF was prepared and then each strain was inoculated onto a dish using a 10 μl calibrated loop.

The readings were carried out after 24 and 48 hours of incubation at 37° C.

In total, 15 Staphylococcus strains were tested: 5 MRSAs and 2 MSSAs, 4 S. epidermidis, 1 S. cohnii, 4 S. sciuri and 2 S. saprophyticus.

The results are recorded in Table 7 hereafter:

TABLE 7
Detection of MRSAs on a selective medium by
means of their β-ribosidase activity

| Strains | IT | Growth | Strength | Coloration |
|---|---|---|---|---|
| MRSA | 24 h | − | — | Turquoise |
| 060 | 48 h | + | 3 | |
| MRSA | 24 h | + | 1 | Turquoise |
| 024 | 48 h | + | 3 | |
| MRSA | 24 h | + | 2.5 | Turquoise |
| 08 060 | 48 h | + | 3 | |
| MRSA | 24 h | + | 2.5 | Turquoise |
| 012 | 48 h | + | 3 | |
| MRSA | 24 h | + | — | Turquoise |
| 023 | 48 h | + | 3 | |
| MSSA | 24 h | − | — | — |
| 049 | 48 h | − | — | |
| MSSA | 24 h | − | — | — |
| 061 | 48 h | − | — | |
| S. epidermidis | 24 h | − | — | — |
| 061 | 48 h | − | — | |
| S. epidermidis | 24 h | − | — | — |
| 164 | 48 h | − | — | |
| S. epidermidis | 24 h | + | — | — |
| 190 | 48 h | + | — | |
| S. epidermidis | 24 h | + | — | — |
| 175 | 48 h | + | — | |
| S. saprophyticus | 24 h | + | — | — |
| 004 | 48 h | + | — | |
| S. saprophyticus | 24 h | − | — | — |
| 061 | 48 h | + | — | |
| S. sciuri | 24 h | − | — | — |
| 029 | 48 h | − | — | |
| S. cohnii | 24 h | + | — | — |
| 040 | 48 h | + | — | |

The results above show that, on a selective medium, inhibiting MSSAs and most of the other Staphylococcus species, it is possible to distinguish MRSAs, which have a turquoise coloration. The other species which grow on this medium do not show any coloration under these culture conditions.

1. A reaction medium for characterizing Staphylococcus aureus, that comprises a metabolic indicator which is a beta-ribosidase substrate in combination with at least one other metabolic indicator and/or at least one metabolic regulator.

2. The reaction medium as claimed in claim 1, according to which said metabolic regulator is chosen from an osidase regulator, a selective compound, or a mixture of selective compounds, an antibiotic or a mixture of antibiotic(s) and/or antifungal agent(s).

3. The reaction medium as claimed in claim 1, according to which said other metabolic indicator is an enzyme substrate.

4. The reaction medium as claimed in claim 3, according to which said enzyme substrate is chosen from a beta-glucosidase and/or beta-galactosidase substrate.

5. The reaction medium as claimed in claim 1, according to which said beta-ribosidase substrate is a beta-ribofuranosidase substrate.

6. The reaction medium as claimed in claim 1, according to which said beta-ribosidase substrate is a substrate chosen from 2-hydroxyphenyl-β-D-ribofuranoside (catechol-β-D-riboside); magenta-β-D-riboside (5-bromo-6-chloro-3-indoxyl-β-D-riboside); dihydroxyflavone-β-D-riboside; X-β-D-riboside (5-bromo-4-chloro-3-indoxyl-β-D-riboside); pink-β-D-riboside (6-chloro-3-indoxyl-β-D-riboside); 6-bromo-3-indoxyl-β-D-riboside; 5-bromo-3-indoxyl-β-D-riboside; 6-fluoro-3-indoxyl-β-D-riboside; alizarin-β-D-riboside; (P)-nitrophenyl-β-D-riboside; 4-methyl-umbelliferyl-β-D-riboside; naphthol-β-D-riboside; dichloroaminophenyl-β-D-riboside.

7. The reaction medium as claimed in claim 6, according to which said beta-ribofuranosidase substrate is 2-hydroxyphenyl-beta-D-ribofuranoside.

8. The reaction medium as claimed in claim 6, according to which said beta-ribosidase substrate is 5-bromo-4-chloro-3-indoxyl-β-D-riboside.

9. A method for detecting and/or identifying Staphylococcus aureus, the method comprising: providing a metabolic indicator which is a beta-ribosidase substrate in combination with at least one other metabolic indicator and/or at least one metabolic regulator.

10. The method as claimed in claim 9, according to which said beta-ribosidase substrate is a beta-ribofuranosidase substrate.

11. The method as claimed in claim 9, in which said beta-ribosidase substrate is a substrate chosen from: 2-hydroxyphenyl-β-D-ribofuranoside (catechol-β-D-riboside); magenta-β-D-riboside (5-bromo-6-chloro-3-indoxyl-β-D-riboside); dihydroxyflavone-β-D-riboside; X-β-D-riboside (5-bromo-4-chloro-3-indoxyl-β-D-riboside); pink-β-D-riboside (6-chloro-3-indoxyl-β-D-riboside); 6-bromo-3-indoxyl-β-D-riboside; 5-bromo-3-indoxyl-β-D-riboside; 6-fluoro-3-indoxyl-β-D-riboside; alizarin-β-D-riboside; (P)-nitrophenyl-β-D-riboside; 4-methylumbelliferyl-β-D-riboside; naphthol-β-D-riboside; dichloroaminophenyl-β-D-riboside.

12. The method as claimed in claim 11, according to which said substrate is 5-bromo-4-chloro-3-indoxyl-β-D-riboside.

13. The method as claimed in claim 11, according to which said substrate is 2-hydroxyphenyl-beta-D-ribofuranoside.

14. A method for detecting and/or identifying Staphylococcus aureus, comprising the following steps:
a) providing a reaction medium as claimed in claim 1,
b) inoculating the medium with a biological sample to be tested,
c) allowing for incubation, and
d) characterizing the presence of Staphylococcus aureus.

15. The method as claimed in claim 14, further comprising a confirmation step.
